(19) United States
(12) Patent Application Publication
(10) Pub. No.: US 2022/0331221 A1
(43) Pub. Date: Oct. 20, 2022

(54) HAIR LOSS TREATMENT

(71) Applicant: Adegen LLC, Sheridan, WY (US)

(72) Inventor: John Goss, Grove City, OH (US)

(21) Appl. No.: 17/724,547

(22) Filed: Apr. 20, 2022

Related U.S. Application Data
(60) Provisional application No. 63/176,958, filed on Apr. 20, 2021.

(51) Int. Cl. A61K 8/49; A61K 8/9789; A61Q 5/02; A61Q 7/00; A61K 8/67; A61K 8/36; A46B 9/02

(52) U.S. Cl. CPC A61K 8/4953; A61K 8/9789; A61Q 5/02; A61Q 7/00; A61K 8/673; A61K 8/36; A46B 9/023; A61K 2800/88; A46B 2200/104

(57) ABSTRACT

Described herein is a hair-loss prevention kit comprising a hair-loss composition comprising caffeine; and a brush comprising a body having a first major surface opposite a second major surface and a plurality of bristles extending from the second major surface of the body.

### CROSS-REFERENCE TO RELATED APPLICATIONS

This application claims the benefit of U.S. Provisional Application No. 63/176,958, filed on Apr. 20, 2021. The disclosures of the above applications are incorporated herein by reference.

### BACKGROUND

Thinning hair and hair loss (androgenic alopecia) is the result of blood flow restriction to the hair follicle. Previously, hair loss was treated by a topical composition that comprising minoxidil. Problems with these previous approaches to hair loss is that minoxidil is strictly a vasodialtor and assist with increasing blood flow to the hair but it doesn't do anything to address the actual problem that is causing the restriction. Therefore, the need exists for an improved approach to treating hair loss.

### BRIEF SUMMARY

Some embodiments of the present invention include a hair-loss prevention kit comprising: a hair-loss composition comprising caffeine; and a brush comprising a body having a first major surface opposite a second major surface and a plurality of bristles extending from the second major surface of the body.

Other embodiments of the present invention include a method of treating hair loss comprising: a) applying a hair-loss shampoo to the user's scalp, wherein the hair-loss composition comprises caffeine; b) contacting a user's scalp with a plurality of bristles on a brush to cause both exfoliation of the user's scalp and micro-trauma to the user's scalp.

Other embodiments of the present invention include a brush for the treatment of hair-loss comprising: a body having a first major surface opposite a second major surface; a plurality of bristles extending from the second major surface of the body, the plurality of bristles are present on the brush in a density ranging from about 18 bristle/inch² to about 35 bristle/inch²; wherein each of the plurality of bristles has a central axis that intersects a distal end that is opposite a proximal end, and wherein the distal end has a diameter ranging from about 40 mils inch to about 60 mils.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is top perspective view of a brush configured to treat hair loss according to the present invention;

FIG. 2 is bottom perspective view of the brush according to FIG. 1;

FIG. 3 is a first side view of the brush according to FIG. 1;

FIG. 4 is an alternative side view of the brush according to FIG. 1;

FIG. 5 is top view of the brush according to FIG. 1;

FIG. 6 is bottom view of the brush according to FIG. 1; and

FIG. 7 is cross-sectional view of the brush along line VII-VII as shown in FIG. 1.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top,” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such.

Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Unless otherwise specified, all percentages and amounts expressed herein and elsewhere in the specification should be understood to refer to percentages by weight. The amounts given are based on the active weight of the material. According to the present application, the term “about” means+/−5% of the reference value. According to the present application, the term “substantially free” less than about 0.1 wt. % based on the total of the referenced value.

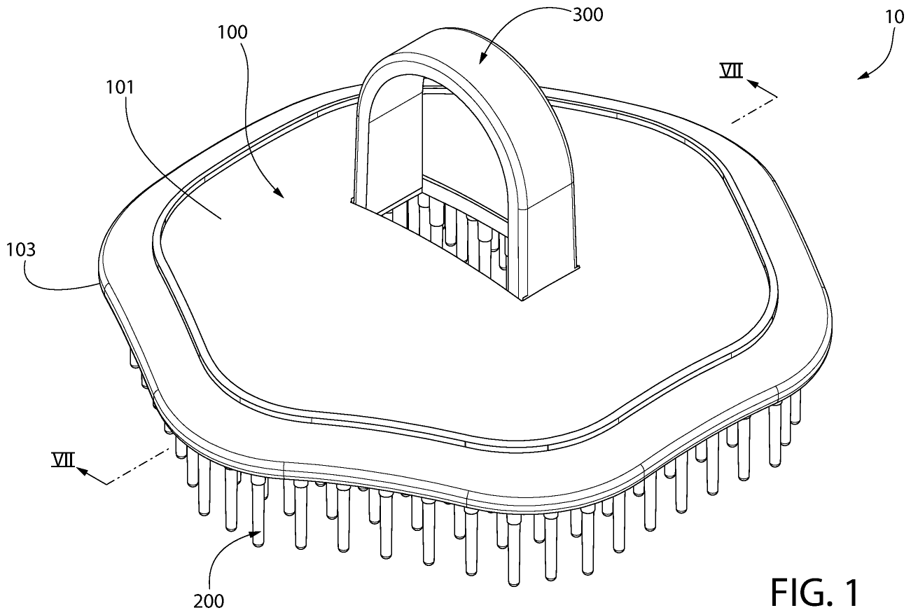

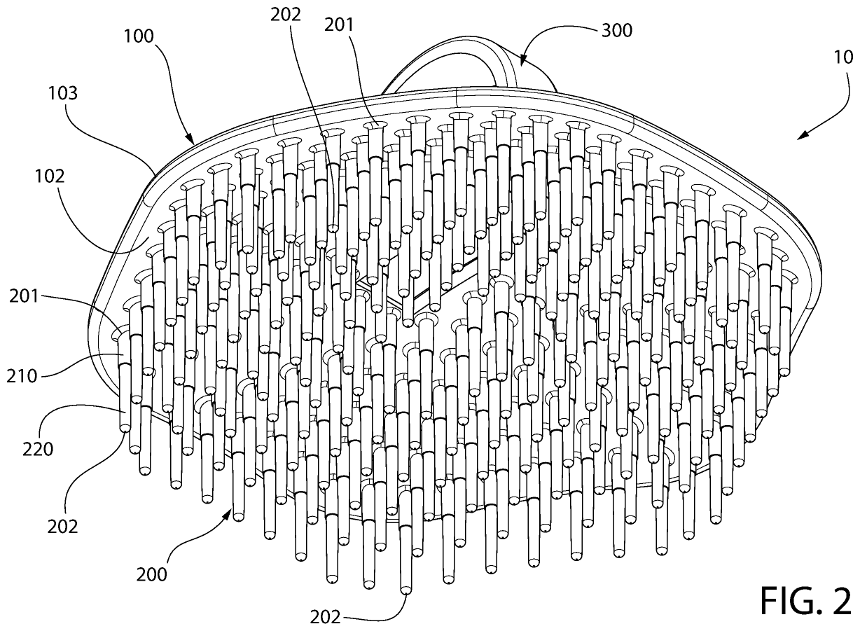

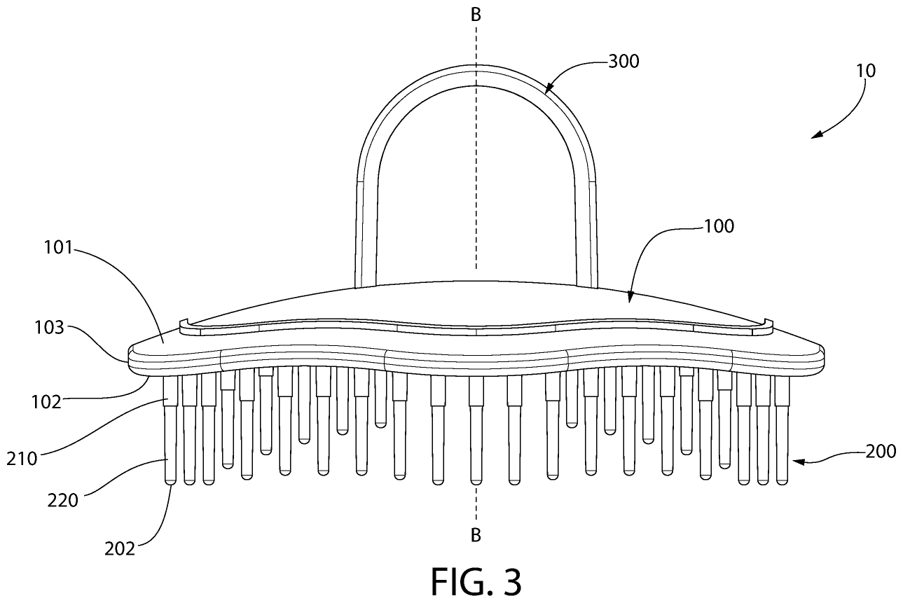

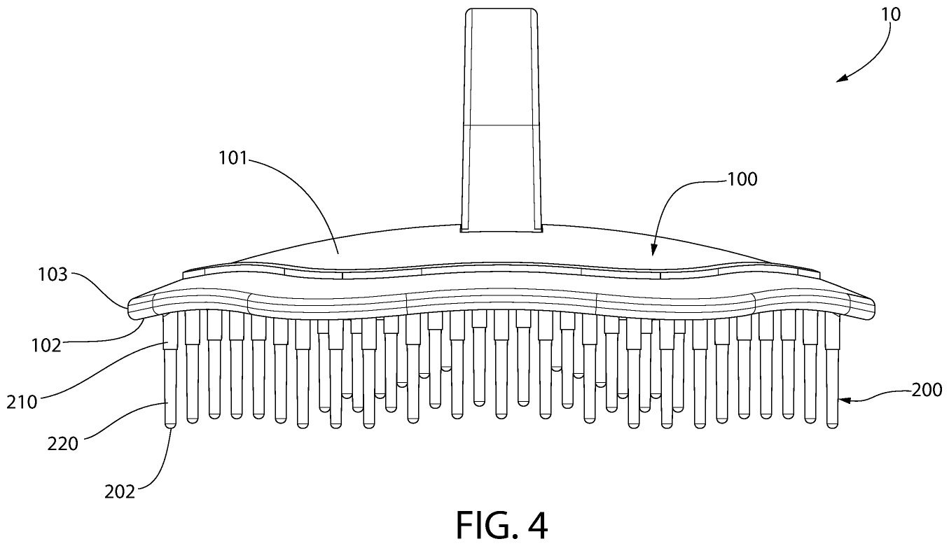

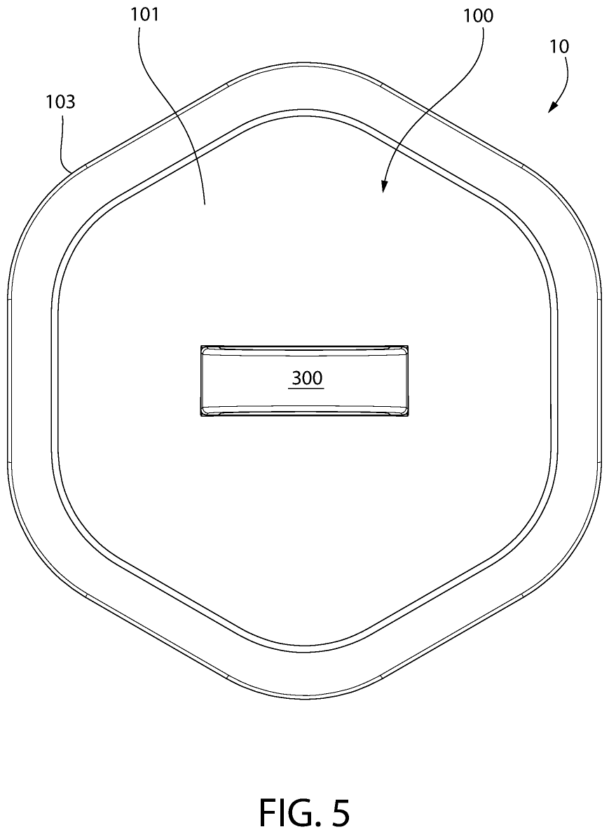

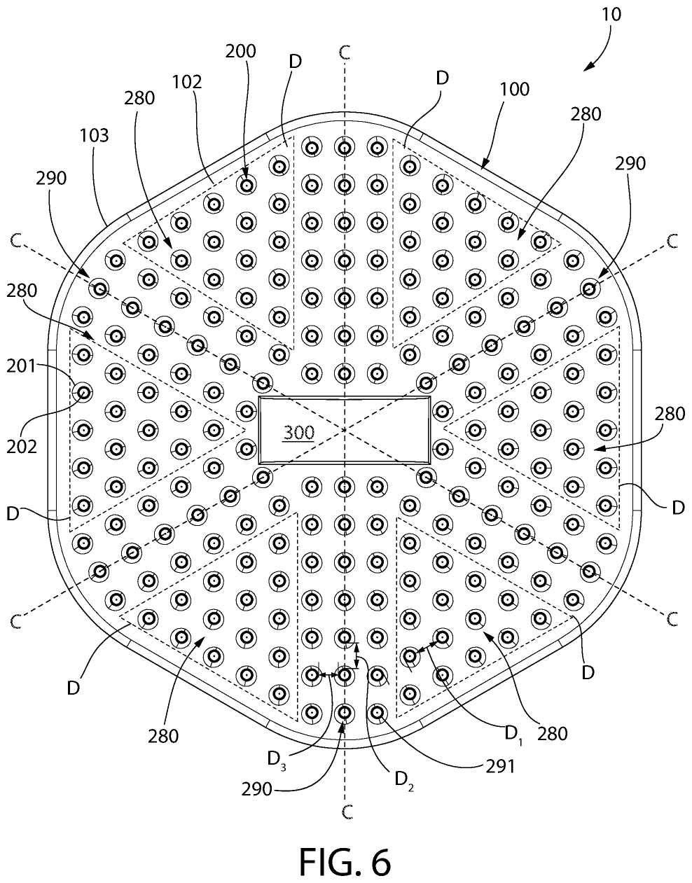

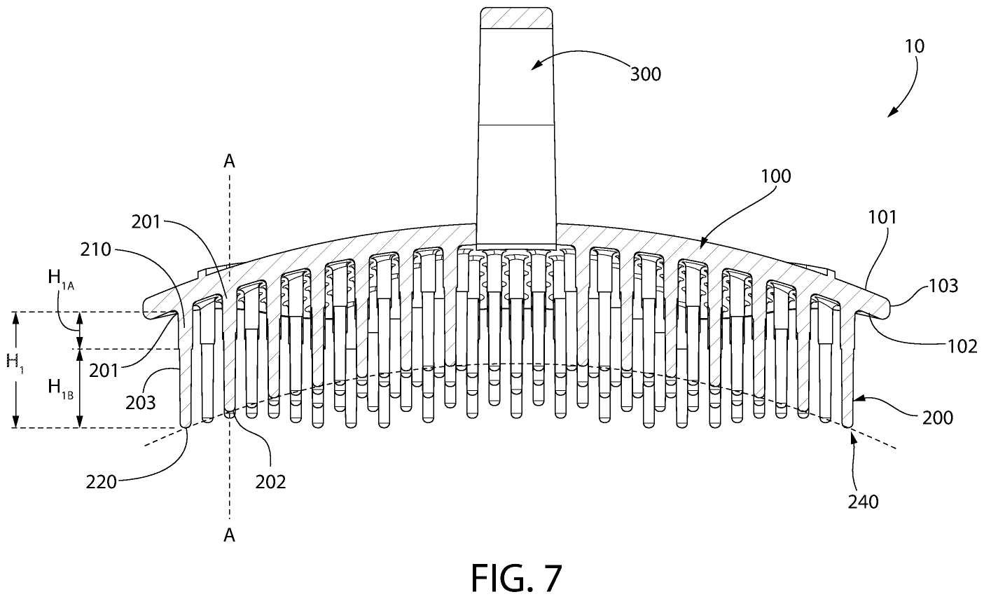

Referring now to FIGS. 1-7, the present invention includes a brush 10 configured for use in the treatment of hair loss. The term “treatment” refers to both the prevention of hair loss as well as the restoration of temporarily lost hair.

The brush 10 may comprise a body 100 and a plurality of bristles 200. The brush 10 may further comprise a gripping element 300. Non-limiting examples of gripping elements 300 include a handle, such as a U-shaped handle.

The body 100 may comprise a first major surface 101 opposite a second major surface 102 and a side surface 103 extending there-between. The side surface 103 may define a perimeter of the body 100. The perimeter of the body 100 as defined by the side surface 103 may be a polygonal shape. The body 100 may have a body central axis B-B that intersects both the first major surface 101 and the second major surface 102. The perimeter may be an undulating shape.

The plurality of bristles 200 may extend from the second major surface 102 of the body 100. The plurality of bristle 200 may be present in a density ranging from about 18 bristle/inch to about 35 bristle/inch²—including all densities and sub-ranges there-between. In some embodiments, the plurality of bristle 200 may be present in a density ranging from about 20 bristle/inch² to about 29 bristle/inch²—including all densities and sub-ranges there-between. The plurality of bristle 200 may be separated from each other by a bristle separation distance ranging from about 0.15 inches to about 0.3 inches—including all distances and sub-ranges there-between. In some embodiments, the plurality of bristle 200 may be separated from each other by a bristle separation distance ranging from about 0.17 inches to about 0.29 inches—including all distances and sub-ranges there-between.

Each of the plurality of bristles 200 may have a diameter ranging from about 30 mils to about 90 mils—including all densities and sub-ranges there-between. In some embodiments, each of the plurality of bristles 200 may have a diameter ranging from about 40 mils to about 80 mils—including all densities and sub-ranges there-between. In some embodiments, each of the plurality of bristles 200 may have a diameter ranging from about 45 mils to about 75 mils—including all densities and sub-ranges there-between.

Each of the plurality of bristles 200 may have a proximal end 201 opposite a distal end 202. The proximal end 201 of each bristle 200 may located immediately adjacent to the second major surface 102 of the body 100. Each of the plurality of bristles 200 may have a central axis A-A which extends longitudinally and intersects both the proximal end 201 and the distal end 202 of the bristle 200.

Each of the plurality of bristles 200 may comprise an outer wall 203 that extends from the proximal end 201 to the distal end 202. The outer wall 203 may be tapered with respect to the central axis A-A, whereby the taper angle ranges from about 0.25° to about 0.75°—including all angles and sub-ranges there-between. In some embodiments, the taper angle is about 0.5°. The taper may exist such that the diameter of each of the plurality of bristles 200 at the proximal end 201 is greater than the diameter of each of the plurality of bristles 200 at the distal end 202.

The central axis A-A of each of the plurality of bristles 200 may be substantially parallel to the central axis B-B of the body 100.

Each of the plurality of bristles may comprise a first section 210 and a second section 220. The first section 210 adjacent to the proximal end 201 and the second section 220 adjacent to the distal end 220. The first section 210 may have a first average diameter and the second section 220 has a second average diameter. The first average diameter may be greater than the second average diameter. The differential between the second average diameter of the second section 220 the first average diameter of the first section 210 may create a stepped tapper for the outer wall 203 of each of the plurality of bristles 200.

Each of the plurality of bristles 200 may have a bristle height H₁ as measured by the length along the central axis A-A between the proximal end 201 and the distal end 202. The bristle height H₁ may range from about 0.35 inch to about 0.65 inch—including all heights and sub-ranges there-between. In some embodiments, the bristle height H₁ may range from about 0.55 inch to about 0.6 inch—including all heights and sub-ranges there-between. In some embodiments, the bristle height H₁ may be about 0.575 inch.

The first section 210 may have a first height H_{1A} and the second section 220 may have a second height H_{1B}. The second height H_{1B} may be greater than the first height H_{1A}. The first height H_{1A} may range from about 0.11 inch to about 0.23 inch—including all heights and sub-ranges there-between. In some embodiments, the first height H_{1A} may range from about 0.19 inch to about 0.21 inch—including all heights and sub-ranges there-between. The second height H_{1B} may range from about 0.22 inch to about 0.42 inch—including all heights and sub-ranges there-between. In some embodiments, the second height H_{1B} may range from about 0.36 inch to about 0.37 inch—including all heights and sub-ranges there-between. A ratio of the second height H_{1B} to the first height H_{1A} may range from about 1.5:1 to about 2:1—including all ratios and sub-ranges there-between. The ratio of the second height H_{1B} to the first height H_{1A} may be about 1.8:1.

The distal end 202 of each of the plurality of bristles 200 may form a convex tip. The diameter of the convex tip may range from about 30 mils to about 70 mils—including all heights and sub-ranges there-between. In some embodiments, the diameter of the convex tip may range from about 40 mils to about 60 mils—including all heights and sub-ranges there-between.

The brush 10 may be an integrally formed structure. The brush 10 may be a single injection molded component. The brush 10 may be formed of a polymer. The polymer may be a polyolefin. In a non-limiting embodiment, the polyolefin may be polypropylene. The polymer may be a thermoplastic polymer. The polymer may have a shore A hardness value ranging from about 70 to about 90—including all hardness values and sub-ranges there-between. In some embodiments, the polymer may have a shore A hardness value of about 80.

The second major surface 102 of the body 100 may be concave in shape. Each of the plurality of bristles 200 may have substantially equal bristle heights H₁ resulting in the distal ends 202 of the plurality of bristles 200 forming a bristle interface 240 that is concave.

The plurality of bristles 200 may be present in two or more bristle regions 280 that are at least partially divided from each other by at least one bristle wall 290. Each of the bristle walls 290 may be linear segment 291 and extend along axis C-C. Each of the bristle walls 290 may comprise a plurality of linear segments 291 that extend parallel along axis C-C. In some embodiments, each of the bristle walls 290 may comprise 2 to 8 linear segments 291 that extend parallel along the axis C-C—including all numbers and sub-ranges there-between. In some embodiments, each of the bristle walls 290 may comprise 2 to 4 linear segments 291 that extend parallel along the axis C-C—including all numbers and sub-ranges there-between. Each of the bristle regions 280 may be generally defined by a perimeter D. Each of the bristle regions may be polygonal in shape.

Each of the bristles 200 within the bristle regions 280 may have a first bristle density and each of the bristles 200 within the bristle walls 290 may have a second bristle density. The first bristle density and the second bristle density may not be equal. The first bristle density may range from about 18 bristle/inch² to about 25 bristle/inch²—including all densities and sub-ranges there-between. The second bristle density ranges from about 25 bristle/inch² to about 35 bristle/inch²—including all densities and sub-ranges there-between.

Each of the bristles 200 within the bristle regions 280 may be separated by a first bristle separation distance D₁. Each of the bristles 200 within the bristle walls 290 may be separated by a second bristle separation distance D₂ as measured in a direction parallel to the axis C-C, and each of the bristles 200 within the bristle walls 290 may be separated by a third bristle separation distance D₃ as measured in a direction orthogonal to the axis C-C. The first bristle separation distance D₁ and the second bristle separation distance D₂ may be equal. The first bristle separation distance D₁ and the third bristle separation distance D₃ may not be equal. The second bristle separation distance D₂ and the third bristle separation distance D₃ may not be equal. The first bristle separation distance D₁ may be greater than the third bristle separation distance D₃. The second bristle separation distance D₂ may be greater than the third bristle separation distance D₃.

The first bristle separation distance D₁ may range from about 0.15 inch to about 0.3 inch—including all densities and sub-ranges there-between. In some embodiments, the first bristle separation distance D₁ may range from about 0.2 inch to about 0.22 inch—including all densities and sub-ranges there-between. The second bristle separation distance D₂ may range from about 0.15 inch to about 0.3 inch—including all densities and sub-ranges there-between. In some embodiments, the second bristle separation distance D₂ may range from about 0.2 inch to about 0.22 inch—including all densities and sub-ranges there-between. The third bristle separation distance D₃ may range from about 0.12 inch to about 0.24 inch—including all densities and sub-ranges there-between. In some embodiments, the third bristle separation distance D₃ may range from about 0.17 inch to about 0.19 inch—including all densities and sub-ranges there-between.

According to other embodiments, the present invention may also include a hair-loss treatment kit comprising the brush 10 and a hair-loss composition. The hair-loss composition may be supplied in any suitable form for dispensing—such as, but not limited to, a releasable container.

The hair-loss composition may comprise caffeine. The caffeine may be present in an amount ranging from about 1 wt. % to about 5 wt. % based on the total weight of the hair-loss composition—including all percentages and sub-ranges there-between. The hair-loss composition may comprise a zinc-containing compound. Non-limiting examples of the zinc-containing compound include zinc salts of pyrrolidone carboxylic acid—also referred to as zinc PCA. The hair-loss composition may comprise niacin. The hair-loss composition may comprise menthol. The hair-loss composition may comprise a liquid carrier—such as water.

The hair-loss composition may be a topical treatment. The hair-loss composition may be a shampoo.

According to other embodiments, the present invention may also include a method of treating hair loss. The method may comprise applying the hair-loss composition a user's scalp, followed by contacting the user's scalp with the plurality of bristles 200 on the brush 10. The distal end 202 of each of the plurality of bristles 200 may contact the user's scalp to cause both exfoliation of the user's scalp and micro-trauma to the user's scalp. The concave bristle interface 240 may contact the scalp of the user.

It has been discovered that the brush 10 is capable of contacting a user's scalp and easily passing through the hair (if present) without pulling or damaging that hair, but also providing exfoliation necessary to removes dead skin, dandruff, and sebum present on the epidermis that are clogging the pores while also causing micro-trauma and micro-damage to the scalp and epidermis, which has the benefit of increasing the absorption and effectiveness of the hair-loss composition, thereby triggering the body's natural healing response and it sends blood flow to the area which is then increasing oxygen and nutrients to the hair follicle and resulting in a reduction of hair loss and an increase in new hair growth and hair thickness in prior thinning or balding areas.

1. A hair-loss treatment kit comprising:
a hair-loss composition comprising caffeine; and
a brush comprising a body having a first major surface opposite a second major surface and a plurality of bristles extending from the second major surface of the body.

2. The hair-loss treatment kit according to claim 1, wherein the second major surface has a concave shape.

3. The hair-loss treatment kit according to claim 1, wherein each of the plurality of bristles has a central axis that intersects a distal end that is opposite a proximal end, and where each of the plurality of bristles has an outer wall that is tapered between the distal end and the proximal end;
wherein a taper angle is formed between the outer wall and the central axis, the taper angle ranging from about 0.25° to about 0.75°;
wherein the distal end forms a convex tip; and
wherein the distal end has a diameter ranging from about 30 mils to about 70 mils.

4. (canceled)

5. The hair-loss treatment kit according to claim 3, wherein the outer wall has a stepped taper;
wherein each of the bristles comprise a first section and a second section, the first section adjacent to the proximal end and the second section adjacent to the distal end, wherein first section has a first average diameter and the second section has a second average diameter, the first average diameter being greater than the second average diameter; and
wherein the first section has a first height and the second section has a second height, wherein the second height is greater than the first height.

6.-8. (canceled)

9. The hair-loss treatment kit according to claim 1, wherein the brush is formed of thermoplastic polypropylene having a hardness ranging from about 70 to 90 on the Shore A hardness scale.

10.-13. (canceled)

14. The hair-loss treatment kit according to claim 1, wherein the plurality of bristles are present on the brush in a density ranging from about 18 bristle/inch² to about 35 bristle/inch².

15. (canceled)

16. The hair-loss treatment kit according to claim 1, wherein the caffeine is present in an amount ranging from about 1 wt. % to about 5 wt. % based on the total weight of the hair-loss composition.

17. The hair-loss treatment kit according to claim 1, wherein the hair-loss composition further comprises at least one of a zinc-containing compound, niacin, menthol, and blends thereof.

18.-20. (canceled)

21. A method of treating hair loss comprising:
a) applying a hair-loss shampoo to the user's scalp, wherein the hair-loss composition comprises caffeine;
b) contacting a user's scalp with a plurality of bristles on a brush to cause both exfoliation of the user's scalp and micro-trauma to the user's scalp.

22. The method of treating hair loss according to claim 21, wherein after step a) and before step b) the hair-loss shampoo is lathered into a foam; and
wherein before step b), the user's scalp is contacted with water having a temperature ranging from about 95° F. to about 112° F.

23.-24. (canceled)

25. A brush for the treatment of hair-loss comprising:
a body having a first major surface opposite a second major surface;
a plurality of bristles extending from the second major surface of the body, the plurality of bristles are present on the brush in a density ranging from about 18 bristle/inch² to about 35 bristle/inch²;
wherein each of the plurality of bristles has a central axis that intersects a distal end that is opposite a proximal end, and wherein the distal end has a diameter ranging from about 30 mils inch to about 70 mils.

26. The brush according to claim 25, wherein the plurality of bristles has an outer wall that is tapered between the distal end and the proximal end;
wherein a taper angle is formed between the outer wall and the central axis, the taper angle ranging from about 0.25° to about 0.75°;
wherein the outer wall has a stepped taper;
wherein the distal end forms a convex tip.

27.-28. (canceled)

29. The brush according to claim 25, wherein each of the bristles comprise a first section and a second section, the first section adjacent to the proximal end and the second section adjacent to the distal end, wherein first section has a first average diameter and the second section has a second average diameter, the first average diameter being greater than the second average diameter;
wherein the first section has a first height and the second section has a second height, wherein the second height is greater than the first height.

30.-31. (canceled)

32. The brush according to claim 25, wherein the brush is formed of thermoplastic polypropylene.

33.-35. (canceled)

36. The brush according to claim 32, wherein the polymer has a hardness ranging from about 70 to 90 on the Shore A hardness scale.

37. The brush according to claim 25, wherein the second major surface has a concave shape.

38. The brush according to claim 25, wherein the plurality of bristles are present in two or more bristle regions that are at least partially divided by at least one bristle wall.

39. The brush according to claim 38, wherein the two or more bristle regions have a first bristle density and the bristle wall has a second bristle density, whereby the first bristle density and the second bristle density are not equal;
wherein the first bristle density ranges from about 18 bristle/inch to about 25 bristle/inch²; and
wherein the second bristle density ranges from about 25 bristle/inch² to about 35 bristle/inch².

40.-41. (canceled)

42. The brush according to claim 38, wherein the two or more bristle regions have a polygonal shape.

43. The brush according to claim 38, wherein the at least one bristle wall is a linear segment.
